SEQUENCE LISTING (36 sequences)

SEQ ID NO: 1 - DNA, 30 bp, Artificial Sequence
  synthetic
tggcgtacatgctagaaagagaattggtcc

SEQ ID NO: 2 - DNA, 32 bp, Artificial Sequence
  synthetic
tgggttaagtgcaacacttcaatataaacact

SEQ ID NO: 3 - DNA, 36 bp, Artificial Sequence
  synthetic
gcgcgcgaggtttctcccagtagccggcgcgcgcgc

SEQ ID NO: 4 - DNA, 33 bp, Artificial Sequence
  synthetic
aactaggaacgctctgtgctttgtgcgagaaca

SEQ ID NO: 5 - DNA, 30 bp, Artificial Sequence
  synthetic
agaccatctgcatttctcgcctcactccag

SEQ ID NO: 6 - DNA, 43 bp, Artificial Sequence
  synthetic
gcgcgcgcacacagggctcatagcagagcagcgagacgcgcgc

SEQ ID NO: 7 - DNA, 30 bp, Artificial Sequence
  synthetic
aagaaacatcccaagcacagcttccagaac

SEQ ID NO: 8 - DNA, 30 bp, Artificial Sequence
  synthetic
ttcttgcgctgctcgtgcggagggtggtgg

SEQ ID NO: 9 - DNA, 37 bp, Artificial Sequence
  synthetic
gcgcgcgacgggcaacaacgatggcggcccgcgcgcg

SEQ ID NO: 10 - DNA, 31 bp, Artificial Sequence
  synthetic
tctgagactcaaatttctggctatactacag

SEQ ID NO: 11 - DNA, 30 bp, Artificial Sequence
  synthetic
acacccaaaccattaattctatattgtaca

SEQ ID NO: 12 - DNA, 47 bp, Artificial Sequence
  synthetic
gcgcgcgggcggctatgtttccgccatggtctgctgctgcgcgcgcg

SEQ ID NO: 13 - DNA, 31 bp, Artificial Sequence
  synthetic
gatgtcatcggactcctcgacgtcgtcctac

SEQ ID NO: 14 - DNA, 30 bp, Artificial Sequence
  synthetic
gatgacctgttttcttttgggttgtcgatg

SEQ ID NO: 15 - DNA, 39 bp, Artificial Sequence
  synthetic
gcgcgcggatgtcatcggactcctcgacgtcggcgcgcg

SEQ ID NO: 16 - DNA, 30 bp, Artificial Sequence
  synthetic
ttccactggcacgcccccaaggaaagatga

SEQ ID NO: 17 - DNA, 32 bp, Artificial Sequence
  synthetic
ttgcttccctaatttagcaagctccacacctt

SEQ ID NO: 18 - DNA, 36 bp, Artificial Sequence
  synthetic
gcgcgcgttccactggcacgcccccaagggcgcgcg

SEQ ID NO: 19 - DNA, 30 bp, Artificial Sequence
  synthetic
agcggcggttgctctggttgaagccaagca

SEQ ID NO: 20 - DNA, 30 bp, Artificial Sequence
  synthetic
tatggagctctgaactgactgcactgcttt

SEQ ID NO: 21 - DNA, 45 bp, Artificial Sequence
  synthetic
gcgcgcgaaatcacagcggcggttgctctggttgaagcgcgcgcg

SEQ ID NO: 22 - DNA, 32 bp, Artificial Sequence
  synthetic
actgagacacccccacgagcaccggaacaaga

SEQ ID NO: 23 - DNA, 30 bp, Artificial Sequence
  synthetic
tgtgatgatggttgggcgatgggctgtttc

SEQ ID NO: 24 - DNA, 47 bp, Artificial Sequence
  synthetic
gcgcgcggagacacccccacgagcaccggaacaagacccggcgcgcg

SEQ ID NO: 25 - DNA, 30 bp, Artificial Sequence
  synthetic
gaaatccagaacacacaagtcaagaggaaa

SEQ ID NO: 26 - DNA, 30 bp, Artificial Sequence
  synthetic
ttcctcttgaccggatgttgtatagacttg

SEQ ID NO: 27 - DNA, 37 bp, Artificial Sequence
  synthetic
gcgcgcgtccactcgaccacctccgaaggcgcgcgcg

SEQ ID NO: 28 - DNA, 30 bp, Artificial Sequence
  synthetic
atattaggaatgctccatacattagtagtt

SEQ ID NO: 29 - DNA, 31 bp, Artificial Sequence
  synthetic
tttctatcatttcttctcttagaccaaccat

SEQ ID NO: 30 - DNA, 42 bp, Artificial Sequence
  synthetic
gcgcgcgagtgcaggacctacttcggctcgtgacggcgcgcg

SEQ ID NO: 31 - DNA, 30 bp, Artificial Sequence
  synthetic
agggggatcagcatcatctgggcctggtcg

SEQ ID NO: 32 - DNA, 30 bp, Artificial Sequence
  synthetic
gggagccaaggcccagcaggcgtttaagca

SEQ ID NO: 33 - DNA, 38 bp, Artificial Sequence
  synthetic
gcgcgcggggcacgcagccgggcttgtggttgcgcgcg

SEQ ID NO: 34 - DNA, 30 bp, Artificial Sequence
  synthetic
tactgcaacccttgccctgattgggtgcca

SEQ ID NO: 35 - DNA, 30 bp, Artificial Sequence
  synthetic
gcgctttgcttctgcgccatagggatgccc

SEQ ID NO: 36 - DNA, 41 bp, Artificial Sequence
  synthetic
gcgcgcgggtgccacggaagcccgtgggcgtggagcgcgcg

(19) United States
(12) Patent Application Publication
(10) Pub. No.: US 2024/0360523 A1
(43) Pub. Date: Oct. 31, 2024

(54) Primers, Probes and Kits for Multiplex Detection of Respiratory Pathogen Target Nucleic Acids, and Methods of Use Thereof

(71) Applicant: BOE Technology Group Co., Ltd., Beijing (CN)

(72) Inventor: Mingming ZHAN, Beijing (CN)

(21) Appl. No.: 18/029,087

(22) PCT Filed: Jun. 17, 2022
(86) PCT No.: PCT/CN2022/099556

§ 371 Date: Mar. 28, 2023

(51) Int. Cl. C12Q 1/70

(52) U.S. Cl. CPC C12Q 1/701; C12Q 2600/16

(57) ABSTRACT

Disclosed in the present application are primers, probes and kits for multiplex detection of respiratory pathogen target nucleic acids, and methods of use thereof. The primers, probes and kits for multiplex detection of respiratory pathogen target nucleic acids and methods of use thereof, include two or more of the following: specific primers and probes for genes of influenza A virus, influenza B virus, human metapneumovirus, human coronavirus HKU1, parainfluenza virus type 1, parainfluenza virus type 2, parainfluenza virus type 3, parainfluenza virus type 4, respiratory syncytial virus type A, respiratory syncytial virus type B, adenovirus and enterovirus EV. The primers, probes, kits and methods provided by the present disclosure realize the simultaneous detection of a plurality of target nucleic acids, greatly improve the detection throughput, significantly reduce the detection time and cost, and improve both specificity and sensitivity of the detection.

### CROSS-REFERENCE TO RELATED APPLICATION

The present application is a U.S. National Phase Entry of International Application No. PCT/CN2022/099556 having an international filing date of Jun. 17, 2022. The above-identified application is hereby incorporated by reference.

### TECHNICAL FIELD

The present disclosure relates to the technical field of biological detection, particularly to a method for detecting nucleic acid of respiratory pathogens, primers in combination with probes for use thereof, and methods of use of the same.

### BACKGROUND

Respiratory tract infection (RTI) is a class of diseases that are most commonly seen in human beings, which can occur in any sex, age or region, and is one of the most important causes of morbidity and death worldwide. The clinical symptoms and signs caused by respiratory tract infection are similar. The clinical manifestations of respiratory tract infection are mainly rhinitis, pharyngitis, laryngitis, tonsillitis and other symptoms, and in severe cases can be tracheitis, bronchitis and pneumonia. However, the treatment methods, curative effects and course of disease are different for infections caused by different pathogens. Therefore, it is of great clinical significance to identify and distinguish specific respiratory virus genes for assisting the diagnosis of respiratory virus infection-related diseases.

### SUMMARY

The following is a summary of the subject matters described herein in detail. The summary is not intended to limit the protection scope of the claims.

Embodiments of the present disclosure provide primers and probes and kits for multiplex detection of respiratory pathogen target nucleic acids, and methods of use thereof.

In an aspect, an embodiment of the present disclosure provides primers and probes for multiplex detection of respiratory pathogen target nucleic acids, wherein, the primers and probes for multiplex detection of respiratory pathogen target nucleic acids comprise upstream primers, downstream primers and probes; and

wherein,

the probes comprise a loop region and a stem region, wherein the loop region is 15 bp to 33 bp in length, and the probes are complementary to target sequences to ensure that the probes are preferentially hybridized to the target sequences, and the AG of the sequences of the probes is greater than −3 kcal/mol;

the distance between the 3′ end of the upstream primers and the 5′ end of the probes is greater than 6 bp; and

different target nucleic acids detected in the same fluorescence channel are amplified by different probes labeled with the same fluorescent group, the melting curves of the amplified products of different target nucleic acids in the same fluorescence channel have different Tm values, and the Tm values in the same fluorescence channel range from 60° C.-80° C., with a gradient of the Tm values controlled at a level of at least 3° C.-5° C.

In some exemplary embodiments, the upstream primers, downstream primers, and probes include two or more of the following: specific primers and probes for genes of influenza A virus, influenza B virus, human metapneumovirus, human coronavirus HKU1, parainfluenza virus type 1, parainfluenza virus type 2, parainfluenza virus type 3, parainfluenza virus type 4,respiratory syncytial virus type A, respiratory syncytial virus type B, adenovirus and enterovirus EV.

In some exemplary embodiments, the upstream primers, downstream primers, and probes include two or more of the following:

upstream primer 1, downstream primer 1 and probe 1 for influenza A virus gene, which are set forth in SEQ ID NO: 1, SEQ ID NO: 2 and SEQ ID NO: 3, respectively;

upstream primer 2, downstream primer 2 and probe 2 for influenza B virus gene, which are set forth in SEQ ID NO: 4, SEQ ID NO: 5 and SEQ ID NO: 6, respectively;

upstream primer 3, downstream primer 3 and probe 3 for human metapneumovirus gene, which are set forth in SEQ ID NO: 7, SEQ ID NO: 8 and SEQ ID NO: 9, respectively;

upstream primer 4, downstream primer 4 and probe 4 for human coronavirus HKU1 gene, which are set forth in SEQ ID NO: 10, SEQ ID NO: 11 and SEQ ID NO: 12, respectively;

upstream primer 5, downstream primer 5 and probe 5 for parainfluenza virus type 1 gene, which are set forth in SEQ ID NO: 13, SEQ ID NO: 14 and SEQ ID NO: 15, respectively;

upstream primer 6, downstream primer 6 and probe 6 for parainfluenza virus type 2 gene, which are set forth in SEQ ID NO: 16, SEQ ID NO: 17 and SEQ ID NO: 18, respectively;

upstream primer 7, downstream primer 7 and probe 7 for parainfluenza virus type 3 gene, which are set forth in SEQ ID NO: 19, SEQ ID NO: 20 and SEQ ID NO: 21, respectively;

upstream primer 8, downstream primer 8 and probe 8 for parainfluenza virus type 4 gene, which are set forth in SEQ ID NO: 22, SEQ ID NO: 23 and SEQ ID NO: 24, respectively;

upstream primer 9, downstream primer 9 and probe 9 for respiratory syncytial virus type A gene, which are set forth in SEQ ID NO: 25, SEQ ID NO: 26 and SEQ ID NO: 27, respectively;

upstream primer 10, the downstream primer 10 and the probe 10 for respiratory syncytial virus type B gene, which are set forth in SEQ ID NO: 28, SEQ ID NO: 29 and SEQ ID NO: 30, respectively;

upstream primer 11, the downstream primer 11 and the probe 11 for the adenovirus gene, which are set forth in SEQ ID NO: 31, SEQ ID NO: 32 and SEQ ID NO: 33, respectively;

upstream primer 12, downstream primer 12 and probe 12 for enterovirus EV gene, which are set forth in SEQ ID NO: 34, SEQ ID NO: 35 and SEQ ID NO: 36, respectively.

In some exemplary embodiments, the upstream primer and the downstream primer may be 30 bp-35 bp in length.

In some exemplary embodiments, the amplicons may be 80 bp-400 bp in length.

In some exemplary embodiments, multiple Gs are avoided at the 5′ end of the primers, and G and C are contained as many as possible at the 3′ end of the primers.

In some exemplary embodiments, the presence of a large number of palindrome structures and primer dimers are avoided in the primer structure.

In some exemplary embodiments, the GC content may be between 40% and 60%.

In some exemplary embodiments, the loop regions of the probes are 15 bp-33 bp in length, fully complementary to and paired with the conserved regions of the amplicons, and the probes are complementary to the region having less secondary structure of the target sequence to ensure that the probes are preferentially hybridized to the target sequence.

In some exemplary embodiments, the probes bind to or near the middle of the amplicons, and the distances between the 3′ ends of the upstream primers and the 5′ ends of the probes are greater than 6 bp.

In some exemplary embodiments, the sequences of the stems are typically 5 bp-7 bp in length, and the Tm values for the stems are not lower than annealing temperatures for the loops, ensuring that the probes not binding to the template are in the stem-loop structures.

In some exemplary embodiments, the GC % may be between 75% and 100%, e.g. CGCACC. . . . GGTGCG, the melting temperature is calculated using mfold Web Server, and the free energy of the hairpin structure is between −3 kcal/mol and 0.5 kcal/mol (1 cal=4.1840 J).

In some exemplary embodiments, direct proximity of the G bases to the fluorescent dyes (typically at the 5′ end of the stem region) is avoided, and the 5′ ends of the stem region may preferably be Cs, since the fluorescence of the fluorescent dyes can be quenched by G bases.

In some exemplary embodiments, the designed probes are checked against the presence of other secondary structures, and Fold Web Server is used to predict the secondary structure model of the designed probe.

In some exemplary embodiments, the reporter label groups can be FAM, HEX, ROX and Cy5, respectively for use in a multiplex detection, and Dabcyl is preferred for quenching groups. Different melting curve peaks are arranged in each channel, and two adjacent peaks differ by 3° C.-5° C. The differentiation of melting curve peaks is realized by Tm values for binding between the loops and the target sequences.

In some exemplary embodiments, the probes have the following characteristics:

1. when detecting a target, a pair of primers and a probe are included at the same time;

2. the sequences of the pair of primers and the probe are perfectly complementary to and paired with the target sequences to be detected, and the regions to be amplified are conserved sequences of the respective targets; further, the sequences of the pair of primers and the probe are only perfectly paired with specific target sequences, and not with other target sequences;

3. the combination of the pair of primers and the probe may include upstream primers, probes and downstream primers, and further the upstream primer can be a segment of oligonucleotide sequences located upstream of a specific probe when being complementary to and paired with the template; the downstream primer can be a segment of oligonucleotide sequences located downstream of the specific probe when being complementary to and paired with the template;

4. further, the sequence information of the upstream primers and the downstream primers may have the following characteristics:

1) the upstream primers may be no less than 30 bp, preferably 30 bp-33 bp, in length;

2) the downstream primers may be no less than 30 bp, preferably 30 bp-33 bp, in length;

3) the amplicons length: fragments amplified by the upstream and downstream primers can be controlled at between 90 bp and 130 bp, preferably within the range of 100 bp-125 bp in length;

5. the probes can be modified oligonucleotides, each in a stem-loop structure as a whole, the two complementary ends form the stem, and the sequence in the middle that is specifically complementary to the respiratory pathogen gene forms the loop; the two ends of the probes are labeled with fluorescent groups and quenching groups, respectively; when the fluorescent group and quenching group of the probe are in close proximity, the fluorescence is quenched, and the spatial configuration of the probe changes after binding with the target molecule, resulting in the recovery of fluorescence;

6. the fluorescent groups can be FAM, VIC, ROX and Cy5, and the quenching groups can be Dabcyl, BHQ1 and BHQ2; when being used, the fluorescent group of each probe can be selected from any of the above FAM, VIC, ROX and Cy5, and the quenching group can be selected from any of the above Dabcyl, BHQ1 and BHQ2; the most preferable schemes are as follows: when one of FAM and VIC is selected as the fluorescent groups, Dabcyl is selected as the quenching group, and when one of ROX and Cy5 is selected as fluorescent groups, BHQ2 is selected as the quenching group;

7. the same fluorescent group can be used to simultaneously label the probes for 2-7 respiratory pathogen genes, preferably, to simultaneously label the probes for four respiratory pathogen genes, for example, the probes for the respiratory pathogens influenza A virus, influenza B virus, human metapneumovirus and human coronavirus HKU1 are labeled with FAM fluorescent groups; the probes for the respiratory pathogens parainfluenza virus type 1-4 are labeled with VIC fluorescent groups, and the probes for the respiratory pathogens respiratory syncytial virus type A, respiratory syncytial virus type B, adenovirus and enterovirus EV are labeled with Cy5 fluorescent groups;

8. there is a specific melting temperature (Tm value) for the loop structure of the probes labeled with the same fluorescent group after binding to the template of respiratory pathogens, the Tm for the loop of the four probes are in a gradient controlled at a level of at least 3° C.-5° C., with an overall range of 55° C.-85°C, preferably 60° C.-80° C.; for example, the Tm values for probe loop structures labeled with FAM fluorescent groups are 67° C., 71° C., 74° C. and 79° C. in turn, and the Tm values for probe loop structures labeled with VIC fluorescent groups are 67° C., 70° C., 74° C. and 78° C. in turn, and the Tm values for probe loop structures labeled with Cy5 fluorescent groups are 68° C., 72° C., 75°° C. and 79° C. in turn;

9. the combination of detection primers and probes can be adjusted in the same channel to achieve the difference of Tm between 3° C. and 5° C., such that the detection of specific targets is completed in the same channel.

In another aspect, an embodiment of the present disclosure provides a kit for multiplex detection of respiratory pathogen target nucleic acids, wherein, the kit for multiplex detection of respiratory pathogen target nucleic acids comprises a nucleic acid amplification reaction solution, a premix, a positive control, and a blank control;

the nucleic acid amplification reaction solution comprises upstream primers, downstream primers and probes; and

wherein,
the probes comprise a loop region and a stem region, wherein the loop region is 15 bp to 33 bp in length, and the probes are complementary to target sequences to ensure that the probes are preferentially hybridized to the target sequences, and the AG of the sequences of the probes is greater than −3kcal/mol;

the distance between the 3′ end of the upstream primers and the 5′ end of the probes is greater than 6 bp; and

different target nucleic acids detected in the same fluorescence channel are amplified by different probes labeled with the same fluorescent group, the melting curves of the amplified products of different target nucleic acids in the same fluorescence channel have different Tm values, and the Tm values in the same fluorescence channel range from 60° C.-80° C., with a gradient of the Tm values controlled at a level of at least 3° C.-5° C.

In some exemplary embodiments, the upstream primers, downstream primers and probes include two or more of the following: specific primers and probes for genes of influenza A virus, influenza B virus, human metapneumovirus, human coronavirus HKU1, parainfluenza virus type 1, parainfluenza virus type 2, parainfluenza virus type 3, parainfluenza virus type 4, respiratory syncytial virus type A, respiratory syncytial virus type B, adenovirus and enterovirus EV.

In some exemplary embodiments, the upstream primers, downstream primers, and probes include two or more of the following:

upstream primer 1, downstream primer 1 and probe 1 for influenza A virus gene, which are set forth in SEQ ID NO: 1, SEQ ID NO: 2 and SEQ ID NO: 3, respectively;

upstream primer 2, downstream primer 2 and probe 2 for influenza B virus gene, which are set forth in SEQ ID NO: 4, SEQ ID NO: 5 and SEQ ID NO: 6, respectively;

upstream primer 3, downstream primer 3 and probe 3 for human metapneumovirus gene, which are set forth in SEQ ID NO: 7, SEQ ID NO: 8 and SEQ ID NO: 9, respectively;

upstream primer 4, downstream primer 4 and probe 4 for human coronavirus HKU1 gene, which are set forth in SEQ ID NO: 10, SEQ ID NO: 11 and SEQ ID NO: 12, respectively;

upstream primer 5, downstream primer 5 and probe 5 for parainfluenza virus type 1 gene, which are set forth in SEQ ID NO: 13, SEQ ID NO: 14 and SEQ ID NO: 15, respectively;

upstream primer 6, downstream primer 6 and probe 6 for parainfluenza virus type 2 gene, which are set forth in SEQ ID NO: 16, SEQ ID NO: 17 and SEQ ID NO: 18, respectively;

upstream primer 7, downstream primer 7 and probe 7 for parainfluenza virus type 3 gene, which are set forth in SEQ ID NO: 19, SEQ ID NO: 20 and SEQ ID NO: 21, respectively;

upstream primer 8, downstream primer 8 and probe 8 for parainfluenza virus type 4 gene, which are set forth in SEQ ID NO: 22, SEQ ID NO: 23 and SEQ ID NO: 24, respectively;

upstream primer 9, downstream primer 9 and probe 9 for respiratory syncytial virus type A gene, which are set forth in SEQ ID NO: 25, SEQ ID NO: 26 and SEQ ID NO: 27, respectively;

upstream primer 10, the downstream primer 10 and the probe 10 for respiratory syncytial virus type B gene, which are set forth in SEQ ID NO: 28, SEQ ID NO: 29 and SEQ ID NO: 30, respectively;

upstream primer 11, the downstream primer 11 and the probe 11 for the adenovirus gene, which are set forth in SEQ ID NO: 31, SEQ ID NO: 32 and SEQ ID NO: 33, respectively;

upstream primer 12, downstream primer 12 and probe 12 for enterovirus EV gene, which are set forth in SEQ ID NO: 34, SEQ ID NO: 35 and SEQ ID NO: 36, respectively.

In some exemplary embodiments, the kit for multiplex detection of respiratory pathogen target nucleic acids may also include a premix, a positive control, a blank control, and the like.

In some exemplary embodiments, the premix may include PCR reaction buffers, dNTPs, Taq enzymes, reverse transcriptases.

In some exemplary embodiments, the positive control is virus-like particles containing specific fragments of the genes of influenza A virus, influenza B virus, human metapneumovirus, human coronavirus HKU1, parainfluenza virus type 1, parainfluenza virus type 2, parainfluenza virus type 3, parainfluenza virus type 4, respiratory syncytial virus type A, respiratory syncytial virus type B, adenovirus and enterovirus EV.

In some exemplary embodiments, the blank control may be sterilized deionized water.

In another aspect, an embodiment of the present disclosure provides a method for multiplex detection of respiratory pathogen target nucleic acids, wherein the method for multiplex detection of respiratory pathogen target nucleic acids comprises amplifying the respiratory pathogen target nucleic acids using upstream primers, downstream primers and probes; and

wherein,

the probes comprise a loop region and a stem region, wherein the loop region is 15 bp to 33 bp in length, and the probes are complementary to target sequences to ensure that the probes are preferentially hybridized to the target sequences, and the AG of the sequences of the probes is greater than −3 kcal/mol;

the distance between the 3′ end of the upstream primers and the 5′ end of the probes is greater than 6 bp; and

different target nucleic acids detected in the same fluorescence channel are amplified by different probes labeled with the same fluorescent group, the melting curves of amplified products of different target nucleic acids in the same fluorescence channel have different Tm values, the Tm values in the same fluorescence channel range from 60° C.-80°C, with a gradient of the Tm values controlled at a level of at least 3° C.-5°C.

In some exemplary embodiments, the upstream primers, downstream primers and probes include two or more of the following: specific primers and probes for genes of influenza A virus, influenza B virus, human metapneumovirus, human coronavirus HKU1, parainfluenza virus type 1, parainfluenza virus type 2, parainfluenza virus type 3, parainfluenza virus type 4,respiratory syncytial virus type A, respiratory syncytial virus type B, adenovirus and enterovirus EV.

In some exemplary embodiments, the upstream primers, downstream primers, and probes include two or more of the following:

upstream primer 1, downstream primer 1 and probe 1 for influenza A virus gene, which are set forth in SEQ ID NO: 1, SEQ ID NO: 2 and SEQ ID NO: 3, respectively;

upstream primer 2, downstream primer 2 and probe 2 for influenza B virus gene, which are set forth in SEQ ID NO: 4, SEQ ID NO: 5 and SEQ ID NO: 6, respectively;

upstream primer 3, downstream primer 3 and probe 3 for human metapneumovirus gene, which are set forth in SEQ ID NO: 7, SEQ ID NO: 8 and SEQ ID NO: 9, respectively;

upstream primer 4, downstream primer 4 and probe 4 for human coronavirus HKU1 gene, which are set forth in SEQ ID NO: 10, SEQ ID NO: 11 and SEQ ID NO: 12, respectively;

upstream primer 5, downstream primer 5 and probe 5 for parainfluenza virus type 1 gene, which are set forth in SEQ ID NO: 13, SEQ ID NO: 14 and SEQ ID NO: 15, respectively;

upstream primer 6, downstream primer 6 and probe 6 for parainfluenza virus type 2 gene, which are set forth in SEQ ID NO: 16, SEQ ID NO: 17 and SEQ ID NO: 18, respectively;

upstream primer 7, downstream primer 7 and probe 7 for parainfluenza virus type 3 gene, which are set forth in SEQ ID NO: 19, SEQ ID NO: 20 and SEQ ID NO: 21, respectively;

upstream primer 8, downstream primer 8 and probe 8 for parainfluenza virus type 4 gene, which are set forth in SEQ ID NO: 22, SEQ ID NO: 23 and SEQ ID NO: 24, respectively;

upstream primer 9, downstream primer 9 and probe 9 for respiratory syncytial virus type A gene, which are set forth in SEQ ID NO: 25, SEQ ID NO: 26 and SEQ ID NO: 27, respectively;

upstream primer 10, the downstream primer 10 and the probe 10 for respiratory syncytial virus type B gene, which are set forth in SEQ ID NO: 28, SEQ ID NO: 29 and SEQ ID NO: 30, respectively;

upstream primer 11, the downstream primer 11 and the probe 11 for the adenovirus gene, which are set forth in SEQ ID NO: 31, SEQ ID NO: 32 and SEQ ID NO: 33, respectively;

upstream primer 12, downstream primer 12 and probe 12 for enterovirus EV gene, which are set forth in SEQ ID NO: 34, SEQ ID NO: 35 and SEQ ID NO: 36, respectively.

The present disclosure has the following beneficial technical effects:

1. due to the advantages of constant temperature amplification and rapid reaction of RPA technology, the whole detection process is shortened to less than 25 min;

2. through the probes labeled with different fluorescent groups and the difference of Tm values of probes binding regions in the same channel, the number of multiple targets that can be detected is “number of channels×number of Tms”, and 12 target genes can be detected at the same time with obvious distinctions;

3. the specific amplification primers of RPA and the specific probes, thus a double specific detection, have high sensitivity and specificity.

Other aspects can be understood upon reading and understanding the drawings and the detailed description.

### BRIEF DESCRIPTION OF DRAWINGS

Accompanying drawings are provided for further understanding of technical solutions of the present disclosure, and constitute a part of the specification, and together with the embodiments of the present disclosure, are intended to explain but not to limit the technical schemes of the present disclosure. Shapes and sizes of one or more components in the drawings do not reflect true scales, and are only intended to schematically describe the contents of the present disclosure.

FIG. 1 shows positive amplification peaks for influenza A virus, influenza B virus, human metapneumovirus and human coronavirus HKU1.

FIG. 2 shows the positive amplification peaks for parainfluenza virus type 1, parainfluenza virus type 2, parainfluenza virus type 3 and parainfluenza virus type 4.

FIG. 3 shows positive amplification peaks for respiratory syncytial virus type A, respiratory syncytial virus type B, adenovirus and enterovirus EV.

FIG. 4 shows the detection peak for the positive quality control of influenza A virus.

FIG. 5 shows the detection peak for the positive quality control of influenza B virus.

FIG. 6 shows the detection peak for the positive quality control of human metapneumovirus.

FIG. 7 shows the detection peak for the positive quality control of human coronavirus HKU1.

FIG. 8 shows the detection peak for the positive quality control of parainfluenza virus type 1.

FIG. 9 shows the detection peak for the positive quality control of parainfluenza virus type 2.

FIG. 10 shows the detection peak for the positive quality control of parainfluenza virus type 3.

FIG. 11 shows the detection peak for the positive quality control of parainfluenza virus type 4.

FIG. 12 shows the detection peak for the positive quality control of respiratory syncytial virus type A.

FIG. 13 shows detection peak for the positive quality control of respiratory syncytial virus type B.

FIG. 14 shows the detection peak for the positive quality control of adenovirus.

FIG. 15 shows the detection peak for the positive quality control of enterovirus EV.

FIG. 16 shows positive amplification peaks for influenza A virus, parainfluenza virus type 2, human metapneumovirus, and parainfluenza virus type 4.

FIG. 17 shows positive amplification peaks for parainfluenza virus type 1, influenza B virus, parainfluenza virus type 3 and human coronavirus HKU1.

FIG. 18 shows positive amplification peaks for respiratory syncytial virus type A, respiratory syncytial virus type B, adenovirus, and enterovirus EV.

### DETAILED DESCRIPTION

To make the objectives, technical solutions, and advantages of the present disclosure clearer, the embodiments of the present disclosure will be described in detail below. It is to be noted that the embodiments in the present disclosure and features in the embodiments may be randomly combined with each other if there is no conflict.

The embodiments of the present disclosure will be described below in combination with the drawings in detail. Implementations may be implemented in a plurality of different forms. Those of ordinary skills in the art may easily understand such a fact that implementation modes and contents may be transformed into one or more forms without departing from the purpose and scope of the present disclosure. Therefore, the present disclosure should not be explained as being limited to only the contents described in following implementation modes. The embodiments in the present disclosure and features in the embodiments may be combined randomly with each other unless conflict.

All technical and scientific terms used herein have the same meanings as understood by those of ordinary skill in the art to which the present disclosure pertains, unless otherwise defined. Although any methods and materials similar or equivalent to those described herein may be used to practice or test the present disclosure, preferred methods and materials are described. For the purposes of the present disclosure the following terms are defined hereinafter.

In this application, unless otherwise explicitly stated, the use of the singular includes the plural. It must be noted that the singular forms “a”, “an” and “the” as used in this specification include plural references, unless the context clearly dictated otherwise. In this application, unless otherwise stated, the use of “or” means “and/or”.

The terms “about” or “approximately” mean to be within an acceptable error range of a particular value, as determined by one of ordinary skill in the art, which will depend in part on how the value is measured or determined, i.e. the limitations of the measurement system. For example, “about” may mean to be within one or more than one standard deviations according to the practice in the art. Alternatively, “about” may mean a range of up to 20%, up to 10%, up to 5%, or up to 1% of a particular value. In other examples, an amount of “about 10” includes 10 and any amount from 9 to 11.

In yet other instances, the term “about” referring to a reference value may also include a range of values of plus or minus 10%, 9%, 8%, 7%, 6%, 5%, 4%, 3%, 2%, or 1% of that value. Alternatively, particularly in relation to biological systems or processes, the term “about” may mean to be within an order of magnitude of a value. When specific values are described in the present application and claims, the term “about” is assumed to mean to be within an acceptable margin of error for the specific values unless otherwise stated.

As use in this specification and one or more claims, the words “comprising” (and any form of comprising, such as “comprise” and “comprises”), “having” (and any form of having, such as “have” and “has”), “including” (and any form of including, such as “include” and “includes”) or “containing” (and any form of containing, such as “contains” and “contain”) are inclusive or open, and do not exclude additional, unlisted elements or method steps. It is contemplated that any of the embodiments discussed in this specification may be implemented with reference to any method or combination of the present disclosure and vice versa. In addition, the combination of the present disclosure may be used to implement the method of the present disclosure.

As used herein, the term “pathogene” (also known as pathogen) is a general designation which generally refers to microorganisms and parasites that can cause diseases. Microorganisms account for the vast majority, including viruses, chlamydia, rickettsia, mycoplasma, bacteria, spirochetes and fungi, etc.; and parasites mainly include protozoa and worms, etc.

As used herein, the detection may be detection for the presence or absence of respiratory pathogens in a sample, i.e. the sample to be detected may be a sample containing a respiratory pathogen or a sample not containing a respiratory pathogen. The detection may be used to screen whether a respiratory pathogen is contained in a sample, for example, to screen an in vitro sample.

### EXAMPLES

At present, the most common molecular diagnosis technology is real-time fluorescence quantitative PCR (qPCR). In the qPCR technology, Taqman hydrolysis probes are added into the conventional PCR reaction system, and get cut under the action of Taq enzyme in the reaction process to release signals, which realize real-time detection of amplification and achieve the purpose of detecting pathogens. With the occurrence of novel coronavirus pandemic and the demand for high-throughput and rapid detection, people always hope to complete the detection of multiple targets at the same time in one tube.

At present, multiplex nucleic acid detection is mainly based on real-time fluorescence quantitative PCR technology. Probes labeled with different fluorescent groups are designed according to the detection channels of fluorescence quantitative PCR instrument, to distinguish and detect different targets. Due to the limitation of detection channels, at most five targets can be detected in one tube (CN212800397U). If more targets are to be detected at the same time, more reactions are required, causing a great increase in detection cost. Patent CN112111566A proposes a method for detecting nucleic acids of multiple respiratory pathogens, wherein, conventional real-time fluorescence quantitative PCR technology is adopted in the amplification stage, and the amplified products for different targets are distinguished using a probe melting curve method, the key of the method is a probe oligonucleotide that can form a stem-loop structure in a single-stranded state, and is labeled with fluorescent groups at both ends, wherein the nucleic acid sequence in the loop is complementary to and paired with the target. When, as the temperature increases, the probe bound to the target sequence by the loop is separated from the target sequence, and the intensity of fluorescence signals vary in such process. Probes are separated from different target sequences at different temperatures. Based on such differences, multiple targets can be detected simultaneously.

In theory, this method can meet the needs of multiplex detection, but has to undergo denaturation, annealing/extension and other processes, which takes a long time (about 120 min) in the conventional PCR method, and cannot meet the clinical needs for rapid detection of various pathogens. Because of its advantages of constant temperature and rapidity, RPA amplification can achieve the purpose of rapid detection on the basis of multiplex detection, which is more helpful to the development of POCT related products.

In order to solve the problems such as long multiplex detection time, limited number of detection targets among others, the present disclosure provides a method for simultaneously detecting a plurality of target nucleic acids. Specific primers are designed for different detection targets. The amplification of the target sequences are completed quickly in one tube using the recombinant polymerase amplification (RPA) technology. Specific probes are designed and labeled with fluorescent groups of different colors. Melting curves plotting the Tm values of probes and target binding sequences in different fluorescence channels and the same fluorescence channel are analyzed. The Tm values of amplification products of different target nucleic acids adjacent in the same fluorescence channel are different by 2° C.-20° C., preferably by 2° C.-8° C., thereby realizing rapid detection of multiple target nucleic acids based on RPA+probe melting curves.

RPA primers are characterized in that:

1. the sequences in target nucleic acids are aligned and conserved regions thereof are selected; 2. the upstream primers and downstream primers are 30 bp-35 bp in length; 3. the amplicons are 80 bp-400 bp in length; 4. multiple Gs are avoided at the 5′ end of primers, and G and C are contained as many as possible at the 3′ end; 5. a large number of palindrome structures and primer dimers are avoided; 6. the content of GC is about 40%-60%.

The probes are characterized in that:

1. the loop regions of the probes are 15 bp-33 bp in length, fully complementary to and paired with the conserved regions of the amplicons, and the probes must be complementary to the region having less secondary structure of the target sequence to ensure that the probes are preferentially hybridized to the target sequence;

2. the probes bind to or near the middle of the amplicons, and the distances between the 3′ ends of the upstream primer and the 5′ ends of the probes are greater than 6 bp;

3. the stem sequences are generally 5 bp-7 bp in length, and the Tm values for the stem are not lower than the annealing temperatures for the loops, so as to ensure that the probes not binding to the template are in the stem-loop structures;

4. the GC % may be between 75% and 100%, e.g. CGCACC. . . . GGTGCG, the melting temperature is calculated using mfold Web Server, and the free energy of the hairpin structure is between −3 kcal/mol and 0.5 kcal/mol (1 cal=4.1840 J);

5. direct proximity of the G bases to the fluorescent dyes (typically at the 5′ end of the stem region) is avoided, and the 5′ ends of the stem region may preferably be Cs, since the fluorescence of the fluorescent dyes can be quenched by G bases.

6. the designed probes are checked against the presence of other secondary structures, and Fold Web Server is used to predict the secondary structure model of the designed probe;

7. the reporter label groups can be FAM, HEX, ROX and Cy5, respectively for use in multiplex detection, and Dabcyl is preferred for quenching groups. Different melting curve peaks were arranged in each channel, and two adjacent peaks differ by 3° C.-5° C. The differentiation of melting curve peaks can be realized by Tm values for binding between the loops and the target sequences.

Primers and probes have the following characteristics:

1. when detecting a target, a pair of primers and a probe are included at the same time;

2. the sequences of the pair of primers and the probe are perfectly complementary to and paired with the target sequences to be detected, and the regions to be amplified are conserved sequences of the respective targets; further, the sequences of the pair of primers and the probe are only perfectly paired with specific target sequences, and not with other target sequences;

3. the combination of the pair of primer and the probe may include upstream primers, probes and downstream primers, and further the upstream primer can be a segment of oligonucleotide sequences located upstream of a specific probe when being complementary to and paired with the template; the downstream primer can be a segment of oligonucleotide sequences located downstream of the specific probe when being complementary to and paired with the template;

4. further, the sequence information of the upstream primers and the downstream primers may have the following characteristics:

1) the upstream primers may be not less than 30 bp, preferably 30 bp-33 bp, in length;

2) the downstream primers may be not less than 30 bp, preferably 30 bp-33 bp, in length;

3) the amplicons length: fragments amplified by the upstream and downstream primers can be controlled at between 90 bp and 130 bp, preferably within the range of 100 bp-125 bp in length;

5. the probes can be modified oligonucleotides, each in a stem-loop structure as a whole, the two complementary ends form the stem, and the sequence in the middle that is specifically complementary to the respiratory pathogen gene forms the loop; the two ends of the probes are labeled with fluorescent groups and quenching groups, respectively; when the fluorescent group and quenching group of the probe are in close proximity, the fluorescence is quenched, and the spatial configuration of the probe changes after binding with the target molecule, resulting in the recovery of fluorescence;

6. the fluorescent groups can be FAM, VIC, ROX and Cy5, and the quenching groups can be Dabcyl, BHQ1 and BHQ2; when being used, the fluorescent group of each probe can be selected from the any of the above FAM, VIC, ROX and Cy5, and the quenching group can be selected from the any of the above Dabcyl, BHQ1 and BHQ2; the most preferable scheme are as follows: when one of FAM and VIC is selected as fluorescent groups, Dabcyl is selected as quenching group, and when one of ROX and Cy5 is selected as fluorescent groups, BHQ2 is selected as quenching group;

7. the same fluorescent group can be used to simultaneously label the probes for 2-7 respiratory pathogen genes, preferably, to simultaneously label the probes of four respiratory pathogen genes, for example, the probes for the respiratory pathogens influenza A virus, influenza B virus, human metapneumovirus and human coronavirus HKU1 are labeled with FAM fluorescent groups; the probes for the respiratory pathogens parainfluenza virus type 1-4 are labeled with VIC fluorescent groups, and the probe for the respiratory pathogens respiratory syncytial virus type A, respiratory syncytial virus type B, adenovirus and enterovirus EV are labeled with Cy5 fluorescent groups;

8. there is a specific melting temperature (Tm value) for the loop structure of the probes labeled with the same fluorescent group after binding to the template of respiratory pathogens, the Tm for the loop parts of the four probes are in a gradient controlled at a level of at least 3° C.-5° C., with an overall range of 55° C.-85°C, preferably 60° C.-80° C.; for example, the Tm values for probe loop structures labeled with FAM fluorescent groups are 67° C., 71° C., 74° C. and 79° C. in turn, and the Tm values for probe loop structures labeled with VIC fluorescent groups are 67° C., 70° C., 74° C. and 78° C. in turn, and the Tm values for probe loop structures labeled with Cy5 fluorescent groups labeled are 68° C., 72° C., 75° C. and 79° C. in turn;

9. the combination of detection primers and probes can be adjusted in the same channel according to the difference of Tm between 3° C. and 5° C., such that the detection of specific targets is completed in the same channel.

### Example 1. Design of the Detection Primers and Probes of the Present Disclosure for Detecting Respiratory Pathogens.

The pathogens detected by the present disclosure are Influenza A (IFV A), Influenza B (IFV B), Respiratory Syncytial Virus (RSV) Types A/B, Parainfluenza Virus (PIV) Types 1-4, Human Metapneumovirus (hMPV), Adenovirus (Adv) and Enterovirus (EV) causing respiratory tract infection. Genes of the above viruses were from plasmids synthesized by Sangon Biotech (Shanghai) Co., Ltd. The following primers and probes were synthesized by Sangon Biotech (Shanghai) Co., Ltd.

The Sequences of primers and probes were shown in Table 1 below.

| | | Detection |
|---|---|---|
| Name | Sequence information | target |
| Upstream primer 1 | TGGCGTACATGCTAGAAAGAGAATTGGTCC | Influenza |
| (SEQ ID NO.1) | | A virus |
| Downstream primer 1 | TGGGTTAAGTGCAACACTTCAATATAAACACT | |
| (SEQ ID NO. 2) | | |
| Probe 1 (SEQ ID NO. | FAM-GCGCGCGAGGTTTCTCCCAGTAGCCGGCG | |
| 3) | CGCGCGC-Dabcyl | |
| Upstream primer 2 | AACTAGGAACGCTCTGTGCTTTGTGCGAGAACA | Influenza |
| (SEQ ID NO. 4) | | B virus |
| Downstream primer 2 | AGACCATCTGCATTTCTCGCCTCACTCCAG | |
| (SEQ ID NO. 5) | | |
| Probe 2 (SEQ ID NO. | FAM-GCGCGCGCACACAGGGCTCATAGCAGAGCAGCGAGA | |
| 6) | CGCGCGC-Dabcyl | |
| Upstream primer 3 | AAGAAACATCCCAAGCACAGCTTCCAGAAC | Human |
| (SEQ ID NO. 7) | | metapneumo- |
| Downstream primer 3 | TTCTTGCGCTGCTCGTGCGGAGGGTGGTGG | virus |
| (SEQ ID NO. 8) | | |
| Probe 3 (SEQ ID NO. | FAM-GCGCGCGACGGGCAACAACGATGGCGGCCC | |
| 9) | GCGCGCG-Dabcyl | |
| Upstream primer 4 | TCTGAGACTCAAATTTCTGGCTATACTACAG | Human |
| (SEQ ID NO. 10) | | coronavirus |
| Downstream primer 4 | ACACCCAAACCATTAATTCTATATTGTACA | HKU1 |
| (SEQ ID NO. 11) | | |
| Probe 4 (SEQ ID NO. | FAM-GCGCGCG | |
| 12) | GGCGGCTATGTTTCCGCCATGGTCTGCTGCTGC | |
| | GCGCGCG-Dabcyl | |
| Upstream primer 5 | GATGTCATCGGACTCCTCGACGTCGTCCTAC | Parainfluenza |
| (SEQ ID NO. 13) | | virus |
| Downstream primer 5 | GATGACCTGTTTTCTTTTGGGTTGTCGATG | type 1 |
| (SEQ ID NO. 14) | | |
| Probe 5 (SEQ ID NO. | VIC-GCGCGCGGATGTCATCGGACTCCTCGACGTCG | |
| 15) | GCGCGCG-Dabcyl | |
| Upstream primer 6 | TTCCACTGGCACGCCCCCAAGGAAAGATGA | Parainfluenza |
| (SEQ ID NO. 16) | | virus |
| Downstream primer 6 | TTGCTTCCCTAATTTAGCAAGCTCCACACCTT | type 2 |
| (SEQ ID NO. 17) | | |
| Probe 6 (SEQ ID NO. | VIC-GCGCGCGTTCCACTGGCACGCCCCCAAGG | |
| 18) | GCGCGCG-Dabcyl | |
| Upstream primer 7 | AGCGGCGGTTGCTCTGGTTGAAGCCAAGCA | Parainfluenza |
| (SEQ ID NO. 19) | | virus |
| Downstream primer 7 | TATGGAGCTCTGAACTGACTGCACTGCTTT | type 3 |
| (SEQ ID NO. 20) | | |
| Probe 7 (SEQ ID NO. | VIC-GCGCGCG | |
| 21) | AAATCACAGCGGCGGTTGCTCTGGTTGAAGC | |
| | GCGCGCG-Dabcyl | |
| Upstream primer 8 | ACTGAGACACCCCCACGAGCACCGGAACAAGA | Parainfluenza |
| (SEQ ID NO. 22) | | virus |
| Downstream primer 8 | TGTGATGATGGTTGGGCGATGGGCTGTTTC | type 4 |
| (SEQ ID NO. 23) | | |
| Probe 8 (SEQ ID NO. | VIC-GCGCGCG | |
| 24) | GAGACACCCCCACGAGCACCGGAACAAGACCCG | |
| | GCGCGCG-Dabcyl | |
| Upstream primer 9 | GAAATCCAGAACACACAAGTCAAGAGGAAA | Respiratory |
| (SEQ ID NO. 25) | | syncytial |
| Downstream primer 9 | TTCCTCTTGACCGGATGTTGTATAGACTTG | virus |
| (SEQ ID NO. 26) | | type A |
| Probe 9 (SEQ ID NO. | Cy5-GCGCGCGTCCACTCGACCACCTCCGAAGGC | |
| 27) | GCGCGCG-BHQ2 | |
| Upstream primer 10 | ATATTAGGAATGCTCCATACATTAGTAGTT | Respiratory |
| (SEQ ID NO. 28) | | syncytial |
| Downstream primer | TTTCTATCATTTCTTCTCTTAGACCAACCAT | virus |
| 10 (SEQ ID NO. 29) | | type B |
| Probe 10 (SEQ ID | Cy5-GCGCGCGAGTGCAGGACCTACTTCGGCTCGTGACG | |
| NO. 30) | GCGCGCG-BHQ2 | |
| Upstream primer 11 | AGGGGGATCAGCATCATCTGGGCCTGGTCG | Adenovirus |
| (SEQ ID NO. 31) | | |
| Downstream primer | GGGAGCCAAGGCCCAGCAGGCGTTTAAGCA | |
| 11 (SEQ ID NO. 32) | | |
| Probe 11 (SEQ ID | Cy5-GCGCGCGGGGCACGCAGCCGGGCTTGTGGTT | |
| NO. 33) | GCGCGCG-BHQ2 | |
| Upstream primer 12 | TACTGCAACCCTTGCCCTGATTGGGTGCCA | Enterovirus |
| (SEQ ID NO. 34) | | EV |
| Downstream primer | GCGCTTTGCTTCTGCGCCATAGGGATGCCC | |
| 12 (SEQ ID NO. 35) | | |
| Probe 12 (SEQ ID | Cy5-GCGCGCGGGTGCCACGGAAGCCCGTGGGCGTGGA | |
| NO. 36) | GCGCGCG-BHQ2 | |

The loop regions of the probes are shown in bold; the stem regions of the probes are shown in italics and underlined.

The information of primer combinations and detection targets is as follows in Table 2:

| | | Detection |
|---|---|---|
| Combination | Detection target | channel |
| Primer, probe combination 1 | Influenza A virus | FAM |
| Primer, probe combination 2 | Influenza B virus | |
| Primer, probe combination 3 | Human metapneumovirus | |
| Primer, probe combination 4 | Human coronavirus HKU1 | |
| Primer, probe combination 5 | Parainfluenza virus type 1 | VIC |
| Primer, probe combination 6 | Parainfluenza virus type 2 | |
| Primer, probe combination 7 | Parainfluenza virus type 3 | |
| Primer, probe combination 8 | Parainfluenza virus type 4 | |
| Primer, probe combination 9 | Respiratory syncytial | Cy5 |
| | virus type A | |
| Primer, probe combination 10 | Respiratory syncytial | |
| | virus type B | |
| Primer, probe combination 11 | Adenovirus | |
| Primer, probe combination 12 | Enterovirus EV | |

### Example 2. Detecting and Identifying Method for Respiratory Associated Virus

### 1. Sample Preparation

In vitro transcribed RNA of each target was used as positive amplification template, and nuclease-free water was used as negative control sample;

### 2. Preparation of Nucleic Acid Amplification Reaction Solution

The nucleic acid amplification reaction solution comprises PCR reaction solution and mixed solution of primers and probes

The PCR reaction solution was prepared as follows in Table 3:

| | Concentrations of | |
|---|---|---|
| Reaction components | working solutions | |
| T4 UvsX Recombinase (SBS | 120 | ng/μL |
| Genetech Co., Ltd., Beijing, | | |
| China) | | |
| T4 UvsY Protein (SBS | 60 | ng/μL |
| Genetech Co., Ltd., Beijing, | | |
| China) | | |
| Single chain binding protein | 600 | ng/μL |
| (New England Biolabs) | | |
| Bsu DNA polymerase (New | 5 | U/reaction |
| England Biolabs) | | |
| Reverse transcriptase | 4 | U/reaction |
| (Promega) | | |
| dNTPs (TIANGEN | 200 | μm |
| BIOTECH (BEIJING) Co., | | |
| LTD.) | | |
| Polyethylene glycol 20000 | 5% | |
| (Sigma) | | |
| Dithiothreitol (Sigma) | 2 | mM |
| Phosphocreatine (Macklin | 50 | mM |
| Inc. Co., Ltd., Shanghai, | | |
| China) | | |
| Creatine kinase (150 u/mg) | 100 | ng/μL |
| (Macklin Inc. Co., Ltd., | | |
| Shanghai, China) | | |
| Adenosine Triphosphate | 3 | mM |
| (ATP) (Macklin Inc. Co., | | |
| Ltd., Shanghai, China) | | |
| Trihydroxymethyl | 50 mM | |
| aminomethane (Beyotime | (pH 7.9) | |
| Biotechnology, Shanghai, | | |
| China) | | |
| Potassium acetate (Sigma) | 100 | mM |
| Magnesium acetate (Sigma) | 14 | mM |

The mixed solution of primers and probes was prepared as in Table 4 below.

| Names of primer, probe | Concentration |
|---|---|
| Upstream primer 1 | 420 nM |
| Downstream primer 1 | 420 nM |
| Probe 1 | 200 nM |
| Upstream primer 2 | 360 nM |
| Downstream primer 2 | 360 nM |
| Probe 2 | 150 nM |
| Upstream primer 3 | 300 nM |
| Downstream primer 3 | 300 nM |
| Probe 3 | 200 nM |
| Primer 4 | 420 nM |
| Downstream primer 4 | 300 nM |
| Probe 4 | 150 nM |
| Upstream primer 5 | 300 nM |
| Downstream primer 5 | 300 nM |
| Probe 5 | 200 nM |
| Upstream primer 6 | 420 nM |
| Downstream primer 6 | 420 nM |
| Probe 6 | 200 nM |
| Upstream primer 7 | 240 nM |
| Downstream primer 7 | 240 nM |
| Probe 7 | 150 nM |
| Upstream primer 8 | 420 nM |
| Downstream primer 8 | 420 nM |
| Probe 8 | 200 nM |
| Upstream primer 9 | 360 nM |
| Downstream primer 9 | 420 nM |
| Probe 9 | 150 nM |
| Upstream primer 10 | 420 nM |
| Downstream primer 10 | 300 nM |
| Probe 10 | 200 nM |
| Upstream primer 11 | 420 nM |
| Downstream primer 11 | 360 nM |
| Probe 11 | 150 nM |
| Upstream primer 12 | 240 nM |
| Downstream primer 12 | 240 nM |
| Probe 12 | 120 nM |

The prepared amplification reagents were transferred to 8-tube strips for PCR, the 8-tube strips were covered and centrifuged instantaneously.

### 3. Detecting on the PCR Instrument

The 8-tube strips were placed in a real-time fluorescence quantitative PCR instrument (Roche LightCycler 480 II). and the following reaction procedure was set as in Table 5 below:

| Steps | Temperature | Time | |
|---|---|---|---|
| RPA reaction | 40° C. | 15 | min |
| Denaturation | 95° C. | 15 | s |
| Annealing | 45° C. | 60 | s |
| Analysis of | 60° C.-95° C. | Fluorescence was collected | |
| melting curves | | once every 0.5° C./s | |
| Instrument cooling | 37° C. | 10 | s |

### 4. Analysis Results

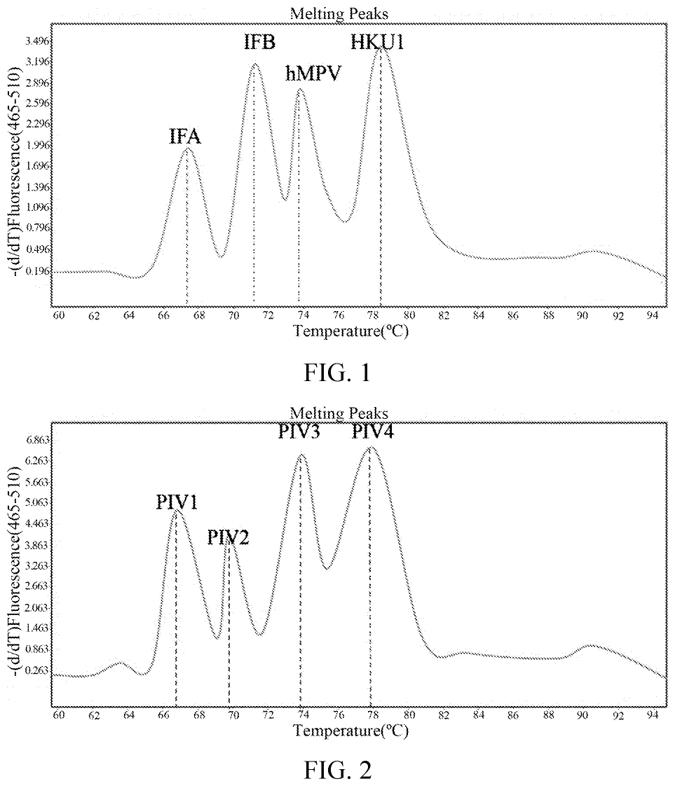

1) In the FAM channel, the peaks of the melting curves were obvious, and there were no other nonspecific amplifications (as shown in FIG. 1):

the characteristic peak appeared in the range of 67° C.±1° C., indicating positive for influenza A virus;

the characteristic peak appeared in the range of 71° C.±1° C., indicating positive for influenza B virus;

the characteristic peak appeared in the range of 74° C.±1° C., indicating positive for human metapneumovirus;

the characteristic peak appeared in the range of 79° C.±1° C., indicating positive for human coronavirus HKU1;

2) In the VIC channel, the peaks of the melting curves were obvious, and there were no other nonspecific amplifications (as shown in FIG. 2):

the characteristic peak appeared in the range of 67° C.±1° C., indicating positive for parainfluenza virus type 1;

the characteristic peak appeared in the range of 70° C.±1° C., indicating positive for parainfluenza virus type 2;

the characteristic peak appeared in the range of 74° C.±1° C., indicating positive for parainfluenza virus type 3;

the characteristic peak appeared in the range of 78° C.±1° C., indicating positive for parainfluenza virus type 4;

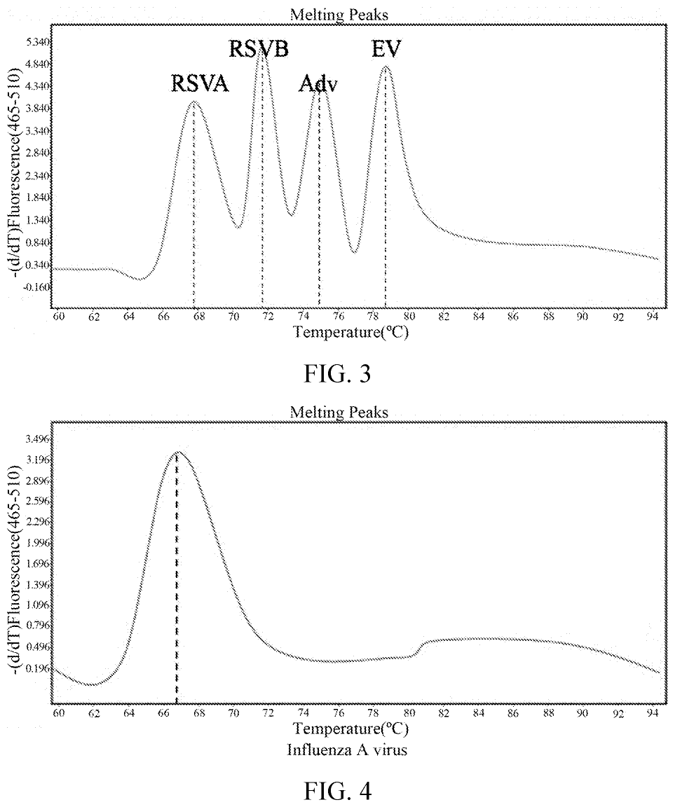

3) In the Cy5 channel, the peaks of the melting curves were obvious, and there were no other nonspecific amplifications (as shown in FIG. 3):

the characteristic peak appeared in the range of 68° C.±1° C., indicating positive for respiratory syncytial virus type A;

the characteristic peak appeared in the range of 72° C.±1° C., indicating positive for respiratory syncytial virus type B;

the characteristic peak appeared in the range of 75° C.±1° C., indicating positive for adenovirus;

the characteristic peak appeared in the range of 79° C.±1° C., indicating positive for enterovirus EV;

Results: the detection time was about 25 min, and the Tm values of 12 viruses were detected, with obvious distinctions between Tm values for different viruses.

### EXAMPLE 3. Test Results from Positive Controls of the Present Disclosure

The plasmids of each target were separately detected, using the combinations of primers and probes in Example 1 of the present disclosure, according to the detection method in Example 2, and the specificity of the detection results of individual targets were evaluated.

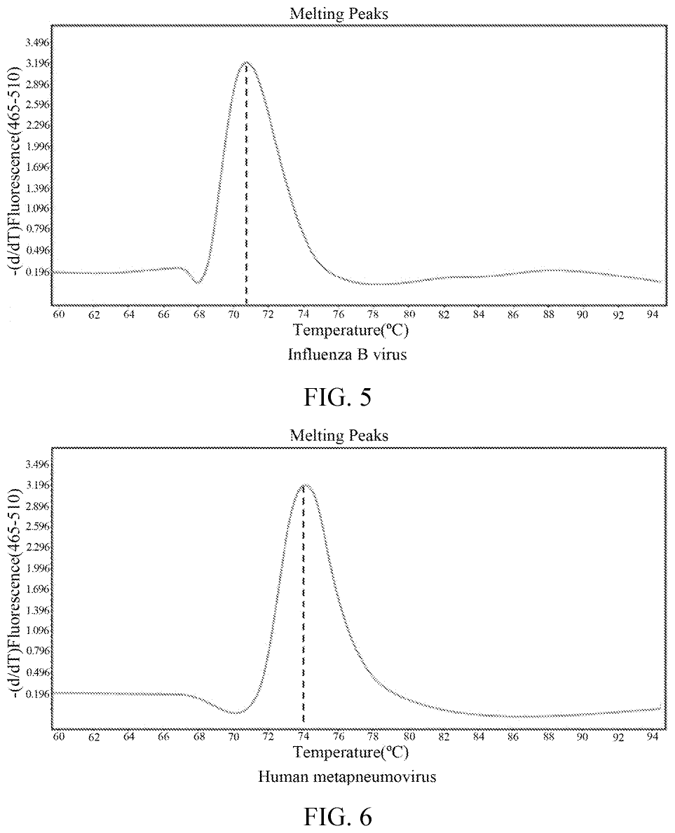

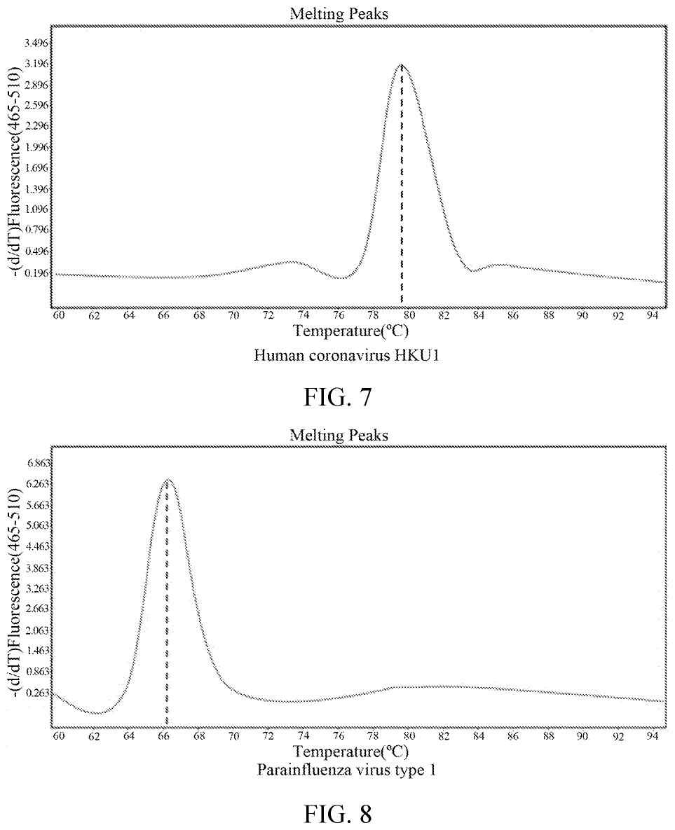

1) The graphs of the detection peaks of the four positive controls in the FAM channel were shown in FIG. 4-7;

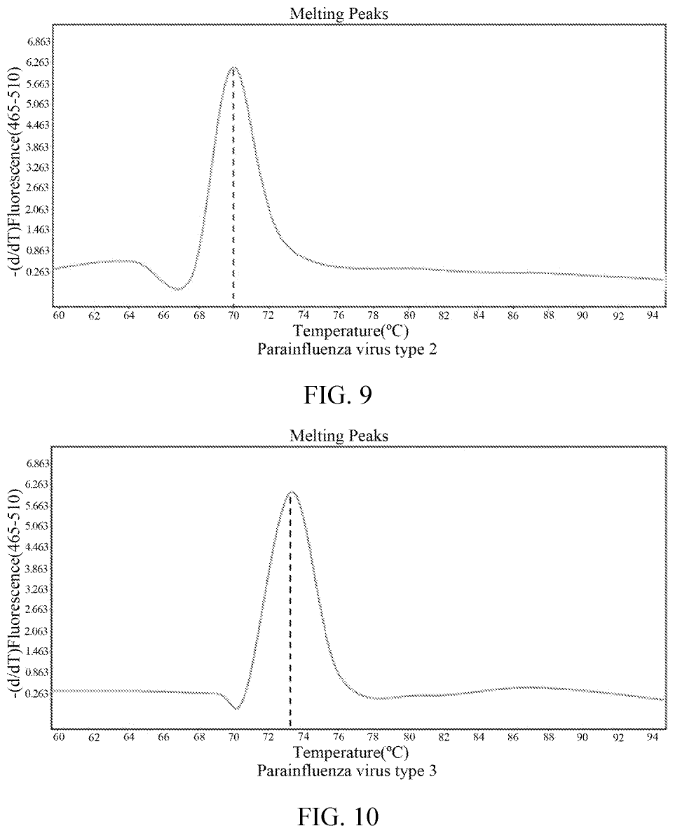

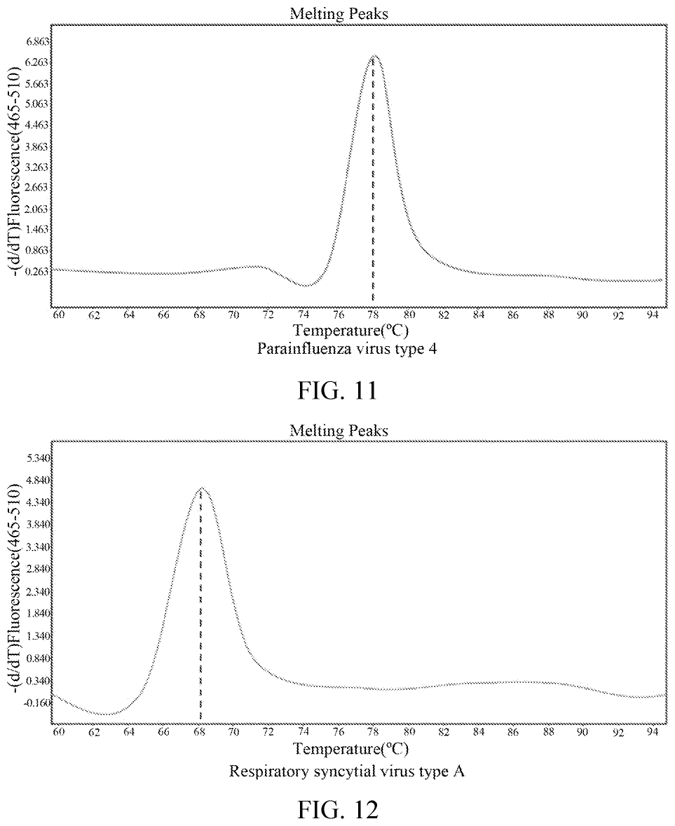

2) the graphs of the detection peaks of the four positive controls in the VIC channel were shown in FIG. 8-11;

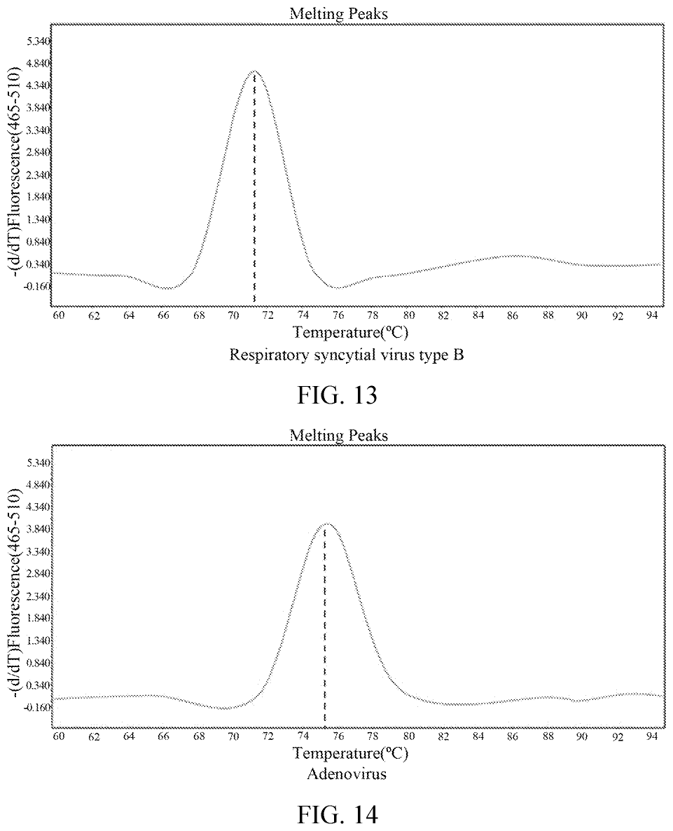

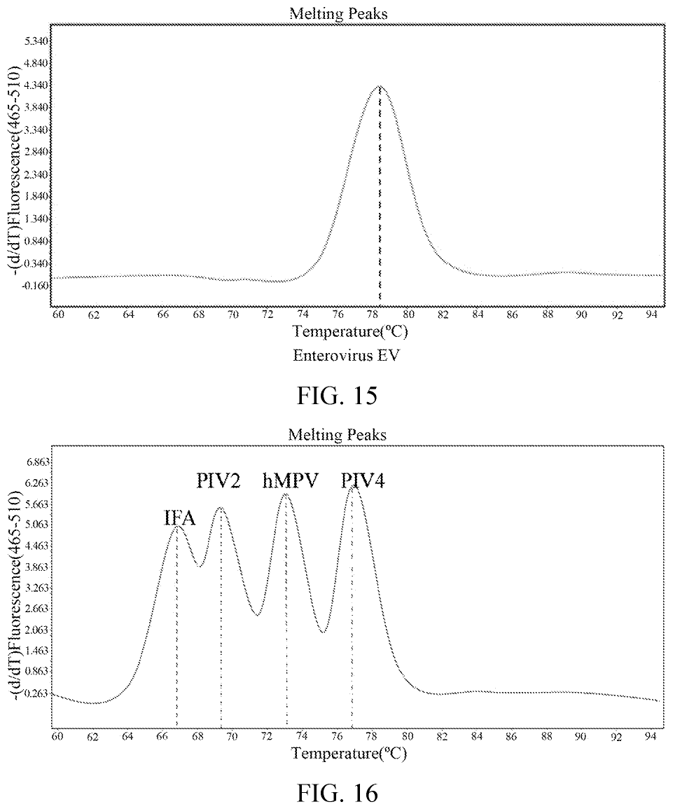

3) the graphs of the detection peaks of the four positive controls in the Cy5 channel were shown in FIG. 12-15;

Summary of Results: all of the Tm values of the specific peaks detected by the positive control standards were in the temperature range described in Example 2, indicating that the reagents detected the respective pathogens stably and the respective pathogens were differentiated obviously.

### Example 4. Detection Sensitivity of the Reagents

The RNA standards transcribed in vitro were successively diluted to 10⁶ copies/mL, 10⁵ copies/mL, 10⁴ copies/mL, 10³ copies/mL, 2×10² copies/mL, and 5×10¹ copies/mL, and amplified according to the detection method described in Example 2. Peaks at specific Tm values corresponding to the respective pathogens indicated positive results for the respective pathogens. The detection results were shown in the table below:

The results of reagent sensitivity verification were as follows in Table 6:

| | 10⁶ | 10⁵ | 10⁴ | 10³ | 2 × 10² | 5 × 10¹ |
|---|---|---|---|---|---|---|
| Virus | copies/mL | copies/mL | copies/mL | copies/mL | copies/mL | copies/mL |
| IFA | + | + | + | + | + | − |
| IFB | + | + | + | + | + | − |
| hMPA | + | + | + | + | + | + |
| HKU1 | + | + | + | + | + | − |
| PIV1 | + | + | + | + | + | − |
| PIV2 | + | + | + | + | + | |
| PIV3 | + | + | + | + | + | + |
| PIV4 | + | + | + | + | + | − |
| RsvA | + | + | + | + | + | + |
| RsvB | + | + | + | + | + | − |
| AdV | + | + | + | + | + | − |
| EV | + | + | + | + | + | − |

Note:
“+” indicates that the results of the tests were positive, and “−” indicates that the results of the tests were negative.

Conclusion: By testing various concentrations of RNA standard samples, ultimately it is determined that the whole detection system worked for all targets and gave positive results when the concentration was 2×10² copies/mL, indicating that, the combinations of primers and probes of the present disclosure can detect targets in the samples even when the targets are at lower concentrations, and thus have a high sensitivity.

### Example 5. Detection Specificity of Different Primers and Probes

1) the combinations of primers and probes were adjusted on the basis of Example 1, that is, making use of the difference between Tm values of different probes to realize the differential detection of each target in different channels. The specific combinations of primers and probes were as follows in Table 7:

| | | Detection |
|---|---|---|
| Combinations | Detection target | channel |
| Primer, probe combination 1 | Influenza A virus | FAM |
| Primer, probe combination 6 | Parainfluenza virus type 2 | |
| Primer, probe combination 3 | Human metapneumovirus | |
| Primer, probe combination 8 | Parainfluenza virus type 4 | |
| Primer, probe combination 5 | Parainfluenza virus type 1 | VIC |
| Primer, probe combination 2 | Influenza B virus | |
| Primer, probe combination 7 | Parainfluenza virus type 3 | |
| Primer, probe combination 4 | Human coronavirus HKU1 | |
| Primer, probe combination 9 | Respiratory syncytial | Cy5 |
| | virus type A | |
| Primer, probe combination 10 | Respiratory syncytial | |
| | virus type B | |
| Primer, probe combination 11 | Adenovirus | |
| Primer, probe combination 12 | Enterovirus EV | |

2) The specific detection method and amount of primers were the same as that in Example 2, and the results of detection were shown in FIG. 16:

the characteristic peak appeared in the range of 67° C.±1° C., indicating positive for influenza A virus;

the characteristic peak appeared in the range of 70° C.±1° C., indicating positive for parainfluenza virus type 2;

the characteristic peak appeared in the range of 74° C.±1° C., indicating positive for human metapneumovirus;

the characteristic peak appeared in the range of 77° C.±1° C., indicating positive for parainfluenza virus type 4;

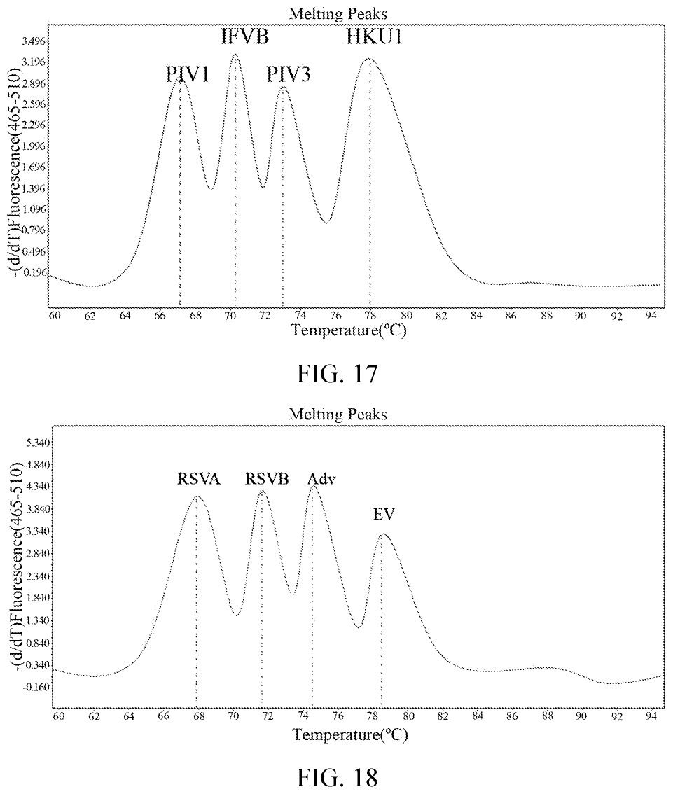

3) the specific detection method and amount of primers were the same as that in Example 2, and the results of detection were shown in FIG. 17:

the characteristic peak appeared in the range of 67° C.±1° C., indicating positive for parainfluenza virus type 1;

the characteristic peak appeared in the range of 70° C.±1° C., indicating positive for influenza B virus was positive;

the characteristic peak appeared in the range of 73° C.±1° C., indicating positive for parainfluenza virus type 3 was positive;

the characteristic peak appeared in the range of 78° C.±1° C., indicating positive for human coronavirus HKU1 was positive;

4) the specific detection method and amount of primers were the same as that in Example 2, and the results of detection were shown in FIG. 18:

the characteristic peak appeared in the range of 68° C.±1° C., indicating positive for respiratory syncytial virus type A;

the characteristic peak appeared in the range of 72° C.±1° C., indicating positive for respiratory syncytial virus type B;

the characteristic peak appeared in the range of 75° C.±1° C., indicating positive for adenovirus;

the characteristic peak appeared in the range of 79° C.±1° C., indicating positive for enterovirus EV was positive;

Conclusion: The combinations of primers and probes can be adjusted and changed according to different Tm values thereof to combine primers and probes for different viruses. The results showed that various targets could be clearly distinguished in different channels, indicating that multiplex detection can be achieved as long as that the Tm values for the primer and probe combinations are within a reasonable range.

1. Primers and probes for multiplex detection of respiratory pathogen target nucleic acids, wherein, the primers and probes for multiplex detection of respiratory pathogen target nucleic acids comprise upstream primers, downstream primers and probes; and
wherein,
the probes comprise a loop region and a stem region, wherein the loop region is 15 bp to 33 bp in length, and the probes are complementary to the target nucleic acids to ensure that the probes are preferentially hybridized to the target nucleic acids, and the AG of the probes is greater than −3 kcal/mol;
the distance between the 3′ end of the upstream primers and the 5′ end of the probes is greater than 6 bp; and
different target nucleic acids detected in the same fluorescence channel are amplified by different probes labeled with the same fluorescent group, the melting curves of the amplified products of different target nucleic acids in the same fluorescence channel have different Tm values, and the Tm values in the same fluorescence channel range from 60° C.-80° C., with a gradient of the Tm values controlled at a level of at least 3° C.-5° C.

2. The primers and probes for multiplex detection of respiratory pathogen target nucleic acids according to claim 1, wherein the upstream primers, the downstream primers and the probes comprise two or more of the following: specific primers and probes for genes of influenza A virus, influenza B virus, human metapneumovirus, human coronavirus HKU1, parainfluenza virus type 1, parainfluenza virus type 2, parainfluenza virus type 3, parainfluenza virus type 4, respiratory syncytial virus type A, respiratory syncytial virus type B, adenovirus and enterovirus EV.

3. The primers and probes for multiplex detection of respiratory pathogen target nucleic acids according to claim 2, wherein the upstream primers, the downstream primers and the probes comprise two or more of the following:
upstream primer 1, downstream primer 1 and probe 1 for influenza A virus gene, which are set forth in SEQ ID NO: 1, SEQ ID NO: 2 and SEQ ID NO: 3, respectively;
upstream primer 2, downstream primer 2 and probe 2 for influenza B virus gene, which are set forth in SEQ ID NO: 4, SEQ ID NO: 5 and SEQ ID NO: 6, respectively;
upstream primer 3, downstream primer 3 and probe 3 for human metapneumovirus gene, which are set forth in SEQ ID NO: 7, SEQ ID NO: 8 and SEQ ID NO: 9, respectively;
upstream primer 4, downstream primer 4 and probe 4 for human coronavirus HKU1 gene, which are set forth in SEQ ID NO: 10, SEQ ID NO: 11 and SEQ ID NO: 12, respectively;
upstream primer 5, downstream primer 5 and probe 5 for parainfluenza virus type 1 gene, which are set forth in SEQ ID NO: 13, SEQ ID NO: 14 and SEQ ID NO: 15, respectively;
upstream primer 6, downstream primer 6 and probe 6 for parainfluenza virus type 2 gene, which are set forth in SEQ ID NO: 16, SEQ ID NO: 17 and SEQ ID NO: 18, respectively;
upstream primer 7, downstream primer 7 and probe 7 for parainfluenza virus type 3 gene, which are set forth in SEQ ID NO: 19, SEQ ID NO: 20 and SEQ ID NO: 21, respectively;
upstream primer 8, downstream primer 8 and probe 8 for parainfluenza virus type 4 gene, which are set forth in SEQ ID NO: 22, SEQ ID NO: 23 and SEQ ID NO: 24, respectively;
upstream primer 9, downstream primer 9 and probe 9 for respiratory syncytial virus type A gene, which are set forth in SEQ ID NO: 25, SEQ ID NO: 26 and SEQ ID NO: 27, respectively;
upstream primer 10, the downstream primer 10 and the probe 10 for respiratory syncytial virus type B gene, which are set forth in SEQ ID NO: 28, SEQ ID NO: 29 and SEQ ID NO: 30, respectively;
upstream primer 11, the downstream primer 11 and the probe 11 for the adenovirus gene, which are set forth in SEQ ID NO: 31, SEQ ID NO: 32 and SEQ ID NO: 33, respectively;
upstream primer 12, downstream primer 12 and probe 12 for enterovirus EV gene, which are set forth in SEQ ID NO: 34, SEQ ID NO: 35 and SEQ ID NO: 36, respectively.

4. A kit for multiplex detection of respiratory pathogen target nucleic acids, wherein, the kit for multiplex detection of respiratory pathogen target nucleic acids comprises a nucleic acid amplification reaction solution, a premix, a positive control and a blank control; and
the nucleic acid amplification reaction solution comprises upstream primers, downstream primers and probes; and
wherein,
the probes comprise a loop region and a stem region, wherein the loop region is 15 bp to 33 bp in length, and the probes are complementary to target nucleic acids to ensure that the probes are preferentially hybridized to the target nucleic acids, and the AG of the probes is greater than −3 kcal/mol;
the distance between the 3 ′ end of the upstream primers and the 5 ′ end of the probes is greater than 6 bp; and
different target nucleic acids detected in the same fluorescence channel are amplified by different probes labeled with the same fluorescencefluorescent group, the melting curves of the amplified products of different target nucleic acids in the same fluorescence channel have different Tm values, and the Tm values in the same fluorescence channel range from 60° C. -80° C., with a gradient of the Tm values controlled at a level of at least 3° C.-5° C.

5. The kit for multiplex detection of respiratory pathogen target nucleic acids according to claim 4, wherein the upstream primers, the downstream primers and the probes comprise two or more of the following:
specific primers and probes for genes of influenza A virus, influenza B virus, human metapneumovirus, human coronavirus HKU1, parainfluenza virus type 1, parainfluenza virus type 2, parainfluenza virus type 3, parainfluenza virus type 4, respiratory syncytial virus type A, respiratory syncytial virus type B, adenovirus and enterovirus EV.

6. The kit for multiplex detection of respiratory pathogen target nucleic acids according to claim 5, wherein the upstream primers, the downstream primers and the probes comprise two or more of the following:
upstream primer 1, downstream primer 1 and probe 1 for influenza A virus gene, which are set forth in SEQ ID NO: 1, SEQ ID NO: 2 and SEQ ID NO: 3, respectively;
upstream primer 2, downstream primer 2 and probe 2 for influenza B virus gene, which are set forth in SEQ ID NO: 4, SEQ ID NO: 5 and SEQ ID NO: 6, respectively;
upstream primer 3, downstream primer 3 and probe 3 for human metapneumovirus gene, which are set forth in SEQ ID NO: 7, SEQ ID NO: 8 and SEQ ID NO: 9, respectively;
upstream primer 4, downstream primer 4 and probe 4 for human coronavirus HKU1 gene, which are set forth in SEQ ID NO: 10, SEQ ID NO: 11 and SEQ ID NO: 12, respectively;
upstream primer 5, downstream primer 5 and probe 5 for parainfluenza virus type 1 gene, which are set forth in SEQ ID NO: 13, SEQ ID NO: 14 and SEQ ID NO: 15, respectively;
upstream primer 6, downstream primer 6 and probe 6 for parainfluenza virus type 2 gene, which are set forth in SEQ ID NO: 16, SEQ ID NO: 17 and SEQ ID NO: 18, respectively;
upstream primer 7, downstream primer 7 and probe 7 for parainfluenza virus type 3 gene, which are set forth in SEQ ID NO: 19, SEQ ID NO: 20 and SEQ ID NO: 21, respectively;
upstream primer 8, downstream primer 8 and probe 8 for parainfluenza virus type 4 gene, which are set forth in SEQ ID NO: 22, SEQ ID NO: 23 and SEQ ID NO: 24, respectively;
upstream primer 9, downstream primer 9 and probe 9 for respiratory syncytial virus type A gene, which are set forth in SEQ ID NO: 25, SEQ ID NO: 26 and SEQ ID NO: 27, respectively;
upstream primer 10, the downstream primer 10 and the probe 10 for respiratory syncytial virus type B gene, which are set forth in SEQ ID NO: 28, SEQ ID NO: 29 and SEQ ID NO: 30, respectively;
upstream primer 11, the downstream primer 11 and the probe 11 for the adenovirus gene, which are set forth in SEQ ID NO: 31, SEQ ID NO: 32 and SEQ ID NO: 33, respectively;
the upstream primer 12, downstream primer 12 and probe 12 of enterovirus EV gene, which are set forth in SEQ ID NO: 34, SEQ ID NO: 35 and SEQ ID NO: 36, respectively.

7. A method for multiplex detection of respiratory pathogen target nucleic acids, wherein method for the multiplex detection of respiratory pathogen target nucleic acids comprises amplifying the respiratory pathogen target nucleic acids using upstream primers, downstream primers and probes; and
wherein,
the probes comprise a loop region and a stem region, wherein the loop region is 15 bp to 33 bp in length, and the probes are complementary to target nucleic acids to ensure that the probes are preferentially hybridized to the target nucleic acids, and the AG of the probes is greater than −3 kcal/mol;
the distance between the 3′ end of the upstream primers and the 5′ end of the probes is greater than 6 bp; and
different target nucleic acids detected in the same fluorescence channel are amplified by different probes labeled with the same fluorescent group, the melting curves of amplified products of different target nucleic acids in the same fluorescence channel have different Tm values, and the Tm values in the same fluorescence channel range from 60° C.-80° C., with a gradient of the Tm values controlled at a level of at least 3° C.-5° C.

8. The method for multiplex detection of respiratory pathogen target nucleic acids according to claim 7, wherein the upstream primers, the downstream primers and the probes comprise two or more of the following:
specific primers and probes for genes of influenza A virus, influenza B virus, human metapneumovirus, human coronavirus HKU1, parainfluenza virus type 1, parainfluenza virus type 2, parainfluenza virus type 3, parainfluenza virus type 4, respiratory syncytial virus type A, respiratory syncytial virus type B, adenovirus and enterovirus EV.

9. The method for multiplex detection of respiratory pathogen target nucleic acids according to claim 8, wherein the upstream primers, the downstream primers and the probes comprise two or more of the following:
upstream primer 1, downstream primer 1 and probe 1 for influenza A virus gene, which are set forth in SEQ ID NO: 1, SEQ ID NO: 2 and SEQ ID NO: 3, respectively;
upstream primer 2, downstream primer 2 and probe 2 for influenza B virus gene, which are set forth in SEQ ID NO: 4, SEQ ID NO: 5 and SEQ ID NO: 6, respectively;
upstream primer 3, downstream primer 3 and probe 3 for human metapneumovirus gene, which are set forth in SEQ ID NO: 7, SEQ ID NO: 8 and SEQ ID NO: 9, respectively;
upstream primer 4, downstream primer 4 and probe 4 for human coronavirus HKU1 gene, which are set forth in SEQ ID NO: 10, SEQ ID NO: 11 and SEQ ID NO: 12, respectively;
upstream primer 5, downstream primer 5 and probe 5 for parainfluenza virus type 1 gene, which are set forth in SEQ ID NO: 13, SEQ ID NO: 14 and SEQ ID NO: 15, respectively;
upstream primer 6, downstream primer 6 and probe 6 for parainfluenza virus type 2 gene, which are set forth in SEQ ID NO: 16, SEQ ID NO: 17 and SEQ ID NO: 18, respectively;
upstream primer 7, downstream primer 7 and probe 7 for parainfluenza virus type 3 gene, which are set forth in SEQ ID NO: 19, SEQ ID NO: 20 and SEQ ID NO: 21, respectively;
upstream primer 8, downstream primer 8 and probe 8 for parainfluenza virus type 4 gene, which are set forth in SEQ ID NO: 22, SEQ ID NO: 23 and SEQ ID NO: 24, respectively;
upstream primer 9, downstream primer 9 and probe 9 for respiratory syncytial virus type A gene, which are set forth in SEQ ID NO: 25, SEQ ID NO: 26 and SEQ ID NO: 27, respectively;
upstream primer 10, the downstream primer 10 and the probe 10 for respiratory syncytial virus type B gene, which are set forth in SEQ ID NO: 28, SEQ ID NO: 29 and SEQ ID NO: 30,respectively;
upstream primer 11, the downstream primer 11 and the probe 11 for the adenovirus gene, which are set forth in SEQ ID NO: 31, SEQ ID NO: 32 and SEQ ID NO: 33, respectively;
upstream primer 12, downstream primer 12 and probe 12 for enterovirus EV gene, which are set forth in SEQ ID NO: 34, SEQ ID NO: 35 and SEQ ID NO: 36, respectively.
